(19) United States
(12) Patent Application Publication
(10) Pub. No.: US 2025/0057633 A1
(43) Pub. Date: Feb. 20, 2025

(54) RFID TAG FOR EXPANDED CAPABILITIES OF A THROMBECTOMY SYSTEM

(71) Applicant: Boston Scientific Scimed, Inc., Maple Grove, MN (US)

(72) Inventors: Mark Alfred Hilse, Somerset, WI (US); Matthew Protas, Blaine, MN (US); Christopher Joel Robinson, Coon Rapids, MN (US); Joel Perez, Corozal (PR); Michael P Schrom, Forest Lake, MN (US); Alyssa Madej, Minnetonka, MN (US); Keval Shah, Maple Grove, MN (US); Corinne Weyrauch, Maple Grove, MN (US); Laszlo Trent Farago, Hudson, WI (US); Taylor Passofaro, St Louis Park, MN (US)

(73) Assignee: Boston Scientific Scimed, Inc., Maple Grove, MN (US)

(21) Appl. No.: 18/801,994

(22) Filed: Aug. 13, 2024

Related U.S. Application Data
(60) Provisional application No. 63/532,482, filed on Aug. 14, 2023.

(51) Int. Cl. A61B 90/98; A61B 17/00; A61B 17/3203; A61B 34/00

(52) U.S. Cl. CPC A61B 90/98; A61B 17/32037; A61B 34/25; A61B 2017/00119; A61B 2017/00199; A61B 2217/007; A61B 2562/0257

(57) ABSTRACT

Thrombectomy systems including a console having a radiofrequency identification (RFID) reader, a fluid inflow pump, the fluid inflow pump driven by the console, a thrombectomy catheter, the fluid inflow pump configured to provide fluid inflow through the thrombectomy catheter, and an RFID tag coupled to a body portion of the fluid inflow pump. When the fluid inflow pump is positioned within the drive unit, the RFID reader may be configured to receive one or more operating parameters from the RFID tag.

### CROSS-REFERENCE TO RELATED APPLICATIONS

This application claims the benefit of U.S. Provisional Patent Application Ser. No. 63/532,482, filed Aug. 14, 2023, which is incorporated herein by reference.

### TECHNICAL FIELD

The disclosure is directed to thrombectomy systems. More particularly, the disclosure is directed to a thrombectomy system with an RFID tag.

### BACKGROUND

Thrombectomy is a procedure for removing thrombus from the vasculature of a patient. Mechanical and fluid-based systems can be used to remove thrombus. With fluid-based systems, an infusion fluid may be infused to a treatment area of a vessel with a catheter to dislodge the thrombus. In some instances, an effluent (e.g., the infusion fluid and/or blood) including the dislodged thrombus may be extracted from the vessel through the catheter. Of the known thrombectomy systems and methods, there is an ongoing need to provide alternative configurations of thrombectomy catheters and systems, as well as methods of operating such thrombectomy systems.

### SUMMARY

This disclosure provides design, material, manufacturing method, and use alternatives for medical devices.

In a first example, a thrombectomy system may comprise a console including a radiofrequency identification (RFID) reader, a fluid inflow pump, the fluid inflow pump driven by the console, a thrombectomy catheter, the fluid inflow pump configured to provide fluid inflow through the thrombectomy catheter, and an RFID tag coupled to a body portion of the fluid inflow pump. When the fluid inflow pump is positioned within the console, the RFID reader may be configured to receive one or more operating parameters from the RFID tag.

Alternatively or additionally to any of the examples above, in another example, the thrombectomy system may further comprise a proximity sensor.

Alternatively or additionally to any of the examples above, in another example, the proximity sensor may be configured to detect the fluid inflow pump is positioned within the console and activate the RFID reader.

Alternatively or additionally to any of the examples above, in another example, the RFID reader may be configured to transfer the one or more operating parameters to a controller of the console.

Alternatively or additionally to any of the examples above, in another example, in response to receiving the one or more parameters the controller may be configured to control one or more components of the thrombectomy system according to the one or more operating parameters.

Alternatively or additionally to any of the examples above, in another example, in response to receiving the one or more parameters the controller may be configured to vary an operational speed of a reciprocating linear actuator.

Alternatively or additionally to any of the examples above, in another example, the operational speed of the reciprocating linear actuator may increase over a period of time.

Alternatively or additionally to any of the examples above, in another example, the operational speed of the reciprocating linear actuator may increase over a stroke length of the reciprocating linear actuator.

Alternatively or additionally to any of the examples above, in another example, in response to receiving the one or more parameters the controller may be configured to display a menu on a user interface, the menu may include one or more user selectable operating parameters.

Alternatively or additionally to any of the examples above, in another example, in response to receiving the one or more parameters the controller may be configured to display a menu on a user interface, the menu may include one more user selectable sounds and/or tones to be assigned by the user for a selected alert and/or notification.

Alternatively or additionally to any of the examples above, in another example, in response to receiving the one or more parameters the controller may be configured to display on a user interface information specific to the thrombectomy catheter and fluid inflow pump.

Alternatively or additionally to any of the examples above, in another example, in response to receiving the one or more parameters the controller may be configured to vary a stroke length of a reciprocating linear actuator of the console.

Alternatively or additionally to any of the examples above, in another example, in response to receiving the one or more parameters the controller may be configured to intermittently activate a roller pump of the console.

Alternatively or additionally to any of the examples above, in another example, in response to receiving the one or more parameters the controller may be configured to reverse an operational direction of a roller pump of the console.

Alternatively or additionally to any of the examples above, in another example, in response to receiving the one or more parameters the controller may be configured to authenticate the fluid inflow pump and thrombectomy catheter.

Alternatively or additionally to any of the examples above, in another example, in response to receiving the one or more parameters the controller may be configured to log usage information within the controller and/or record and transmit operational data to a remote device.

Alternatively or additionally to any of the examples above, in another example, the RFID tag may be disposed within an adhesive label.

In another example, a thrombectomy system may comprise a console including a radiofrequency identification (RFID) reader, a fluid inflow pump, the fluid inflow pump driven by the console, a thrombectomy catheter, the fluid inflow pump configured to provide fluid inflow through the thrombectomy catheter, and a data plate coupled to a body portion of the fluid inflow pump. The data plate may comprise a base layer including an adhesive on a bottom surface thereof, a top layer, and a radiofrequency identification (RFID) tag disposed between the base layer and the top layer. When the fluid inflow pump is positioned within the console, the RFID reader may be configured to receive one or more operating parameters from the RFID tag.

In another example, a data plate for a thrombectomy system may comprise a base layer including an adhesive on a bottom surface thereof, a top layer, a radiofrequency identification (RFID) tag disposed between the base layer and the top layer, and a barcode formed on a top surface of the top layer.

Alternatively or additionally to any of the examples above, in another example, the top layer may be adhesively secured to the RFID tag and a top surface of the base layer.

Alternatively or additionally to any of the examples above, in another example, the base layer and the top layer may be formed from a polyester label material.

Alternatively or additionally to any of the examples above, in another example, the base layer may include an RFID compensation hole extending through a thickness thereof, the RFID compensation hole may be aligned with a chip of the RFID tag.

The above summary of some example embodiments is not intended to describe each disclosed embodiment or every implementation of the disclosure.

### BRIEF DESCRIPTION OF THE DRAWINGS

The disclosure may be more completely understood in consideration of the following detailed description of various embodiments in connection with the accompanying drawings, in which:

FIG. 1 is a perspective view of an illustrative thrombectomy system;

FIG. 2 is a partially exploded perspective view of several components of an illustrative pump/catheter assembly;

FIG. 3 is a top view of an illustrative data plate with a partial cut-out;

FIG. 4 is an exploded perspective view of the illustrative data plate of FIG. 3; and

FIG. 5 is a schematic block diagram of an illustrative control and communications system for a thrombectomy system.

While the disclosure is amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

All numeric values are herein assumed to be modified by the term “about”, whether or not explicitly indicated. The term “about” generally refers to a range of numbers that one of skill in the art would consider equivalent to the recited value (e.g., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

Thrombectomy catheters and systems may be used to remove thrombus, plaques, lesions, clots, etc. from veins or arteries. In some embodiments, a data plate may be included on the pump/catheter assembly, for the inclusion of a means to store, communicate, and/or otherwise determine specifications and/or operational parameters associated with the pump/catheter assembly, the thrombectomy device, the inflow pump, etc., and/or components thereof. In at least some embodiments, a data acquisition device may be configured to communicate with the data plate, the pump/catheter assembly, the thrombectomy device, the inflow pump, etc. to obtain specifications and/or operational parameters associated with the pump/catheter assembly, the thrombectomy device, the inflow pump, etc. and/or components thereof. In some embodiments, the data acquisition device may be configured to communicate with the data plate, the pump/catheter assembly, the thrombectomy device, the inflow pump, etc. to obtain identifying information related thereto which is associated with specifications and/or operational parameters stored in the memory of the user interface and/or the controller. The identifying information may be used by the user interface and/or the controller to access the specifications and/or operational parameters associated with the pump/catheter assembly, the thrombectomy device, the inflow pump, etc. in use that is stored in the memory. However, the number of specifications and/or operational parameters associated with the data plate may be limited. Disclosed herein are thrombectomy catheters and systems that may improve the functionality of the thrombectomy catheter and/or system.

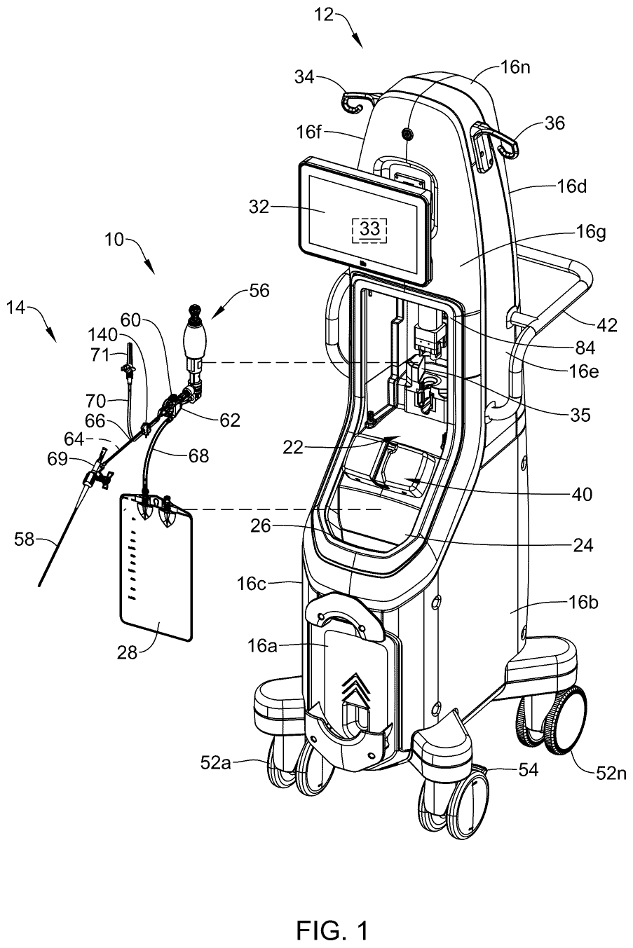

FIG. 1 is a perspective view of an illustrative thrombectomy system 10. The thrombectomy system 10 may include a control console 12, including a drive unit, and a pump/catheter assembly 14. In some instances, the pump/catheter assembly 14 may be a disposable single use device in which a new pump/catheter assembly 14 may be used with the console 12 for each medical procedure. The console 12 may include a housing enclosing the internal structure of the console 12. Shown on the console 12 are a plurality of removable panels 16a-16n about and along the console 12 enclosing the internal structure of the console 12. An illustrative console 12 is described in commonly assigned U.S. Pat. No. 7,935,077, titled THROMBECTOMY CATHETER DEPLOYMENT SYSTEM, the disclosure of which is hereby incorporated by reference. Centrally located in the console 12 and aligned to the lower region of the panel 16g may be automatically opening loading bay door assemblies (not explicitly shown) which open to expose the interior of the console 12 to provide access to a carriage assembly 22 and close during use of the pump/catheter assembly 14. Illustrative loading bay doors and assemblies are described in commonly assigned U.S. Patent Application No. 63/452,517, titled THROMBECTOMY SYSTEM WITH LOADING BAY DOORS, the disclosure of which is hereby incorporated by reference.

The console 12 may include a catch basin or drip tray 24 for collecting fluid leakage from the components of the pump/catheter assembly 14. In some instances, the drip tray 24 may be removable. Other configurations of catch basins are also contemplated. The drip tray 24 and/or a receptacle 26 may collectively support and accommodate an effluent collection bag, such as effluent collection bag 28 of the pump/catheter assembly 14. In other instances, the console 12 may include a different structure, such as a hook for hanging the effluent collection bag 28 from, or a shelf for setting the effluent collection bag 28 on. The effluent waste tube 68 may also be positioned in the roller pump 40 between the tube guides with the effluent collection bag 28 connected to the effluent waste tube 68. The effluent collection bag 28 may be suitably positioned for collecting effluent during the medical procedure. Pump rollers (not shown) of the roller pump 40 may rotatably engage the effluent waste tube 68 to control effluent fluid flow through the effluent waste tube 68 to the effluent collection bag 28.

In instances where the carriage assembly 22 is movable, a carriage assembly activation switch (not explicitly shown) may be provided with the console 12, such as located on a panel 16g, to selectively position the carriage assembly 22 inwardly or outwardly. In other instances, the carriage assembly 22 may be positioned or moved using a control panel and/or user interface 32. A user interface 32, including memory and/or processing capabilities, may be provided with the console 12, such as located at the upper region of the console 12 between the upper regions of the upper side panels 16e and 16f. The user interface 32 may be a guided user interface (GUI) including a touch screen display to allow a user to provide input to the user interface 32 and view information on a same display screen. However, this is not required. In other instances, the user input may be separate from the display screen.

Saline bag hooks 34 and 36 may extend through the panels 16e and 16f to hang saline bags therefrom. The console 12 may include a handle 42 as well as a plurality of wheels 52a-52n and brake pedals 54 for wheel lockage to assist in maneuvering the console 12 by medical personnel.

In FIG. 1, the pump/catheter assembly 14 is shown detached from the console 12. The pump/catheter assembly 14 includes an inflow pump 56 and a thrombectomy device 58. In some embodiments, the inflow pump 56 may be configured to provide fluid inflow through the thrombectomy device 58. During use, a portion of the pump/catheter assembly 14 may be secured within a portion of the console 12. In some embodiments, the pump/catheter assembly 14 may include a bubble trap 60 attached to the inflow pump 56, a connection manifold assembly 62 connected to the bubble trap 60, a fixture 140, an effluent return tube 66 connected between the connection manifold assembly 62 and the thrombectomy device 58, a high-pressure fluid supply tube 64 attached between the output of the inflow pump 56 and the thrombectomy device 58 which may be coaxially arranged inside the effluent return tube 66, a transition fixture 69 between the distal end of the effluent return tube 66 and the proximal end of the thrombectomy device 58, an effluent waste tube 68 connecting the effluent collection bag 28 to the connection manifold assembly 62, and a fluid supply tube 70 having a bag spike 71 connecting a fluid supply bag (e.g., a saline bag) (not explicitly shown) to the connection manifold assembly 62. The fluid supply tube 70 may be in fluid communication with the interior of the bubble trap 60 to provide fluid from the fluid supply bag to the inflow pump 56 and then to the thrombectomy device 58 through the high-pressure fluid supply tube 64.

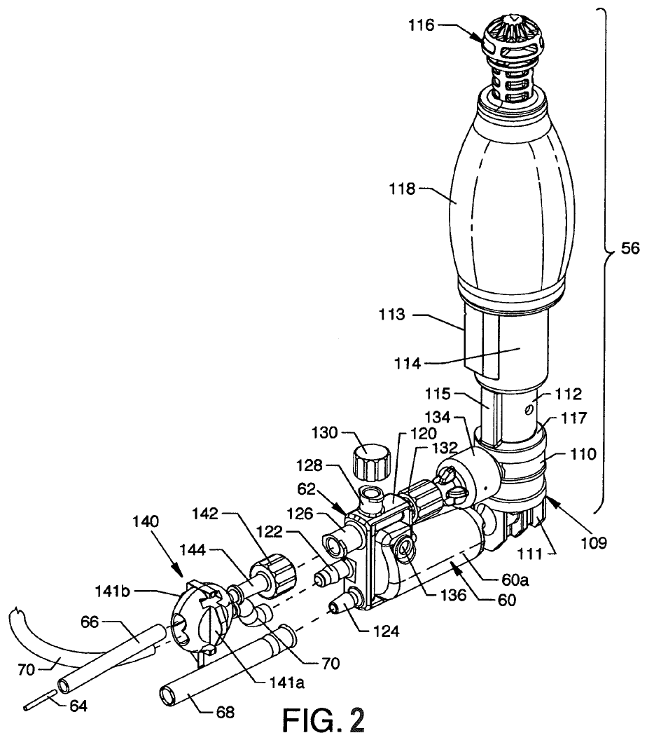

The console 12 may include a reciprocating linear actuator 84 configured to engage a pump piston head 116 (see, for example, FIG. 2) of the inflow pump 56 when the inflow pump 56 is engaged with the carriage assembly 22. The reciprocating linear actuator 84 may be disposed within the interior of the drive unit 12 and may be aligned with the pump piston head 116 (e.g., FIG. 2) when the inflow pump 56 is disposed within the interior of the drive unit 12. The reciprocating linear actuator 84 may be actuated such that reciprocating (e.g., up and down) strokes of the reciprocating linear actuator 84 drive the inflow pump 56 in response to activation of a user activation switch (not explicitly shown) or a control command from a controller 33. In some embodiments, the user activation switch may be a foot switch. In some embodiments, when the user activation switch is depressed, the reciprocating linear actuator 84 and/or the inflow pump 56 is activated and/or runs, and when the user activation switch is released, the reciprocating linear actuator 84 and/or the inflow pump 56 is stopped and/or ceases operation.

The console 12 may include a controller 33 in electronic communication with the user interface 32, the reciprocating linear actuator 84, the inflow pump 56, and/or the thrombectomy device 58. In some cases, the controller 33 may be a part of or otherwise incorporated into the user interface 32. In some embodiments, the console 12 and/or the controller 33 may include a data acquisition device 35. In some embodiments, the data acquisition device 35 may be disposed within the interior of the drive unit 12. For example, the data acquisition device 35 may be positioned on or near the carriage assembly 22. In some embodiments, the data acquisition device 35 may be configured for wireless communication. Other configurations are also contemplated. The data acquisition device 35 may be a radiofrequency identification reader and/or a barcode reader. Other types of data acquisition devices 35 may be used, as desired. In some embodiments, the user activation switch may be in electronic communication with the drive unit 12 and/or the controller 33. In some embodiments, the user activation switch may be in electronic communication with the drive unit 12 and/or the controller 33 via a wire or cable. In some embodiments, the user activation switch may be in electronic communication with the drive unit 12 and/or the controller 33 wirelessly.

FIG. 2 is a partially exploded perspective view of several components of the pump/catheter assembly 14 (e.g., FIG. 1) generally including the inflow pump 56, the bubble trap 60, the connection manifold assembly 62, and the fixture 140. The inflow pump 56 centers about a tubular body 112. Components are located about the lower region of the tubular body 112 and include a base 109 having an upper portion 110 and a lower portion 111 both positioned about the lower region of the tubular body 112. An annular surface 117 is included at the top of the upper portion 110 of the base 109 for intimate contact with capture tabs of the carriage assembly 22 (e.g., FIG. 1) to retain the inflow pump 56 within and/or in engagement with the carriage assembly 22. A top body 114, is positioned about the upper region of the tubular body 112. The base 109 and the top body 114, as well as a connecting panel 115, may be molded or otherwise suitably constructed to encompass the greater part of the tubular body 112, for example.

In some embodiments, a data plate 113 may also be included on the pump/catheter assembly 14, such as on the top body 114 for example, for the inclusion of a barcode, an radiofrequency identification (RFID) tag, a data storage chip, informational displays, etc. to store, communicate, and/or otherwise determine specifications and/or operational parameters associated with the pump/catheter assembly 14, the thrombectomy device 58, the inflow pump 56, etc. and/or components thereof, as will be described in more detail herein. In at least some embodiments, the data acquisition device 35 (e.g., FIG. 1) may be configured to communicate with the data plate 113 (e.g., the barcode, the RFID tag, the data storage chip, etc.), the pump/catheter assembly 14, the thrombectomy device 58, the inflow pump 56, etc. to obtain specifications and/or operational parameters associated with the pump/catheter assembly 14, the thrombectomy device 58, the inflow pump 56, etc., and/or components thereof. In some embodiments, the data acquisition device 35 (e.g., FIG. 1) may be configured to communicate with the data plate 113 (e.g., the barcode, the RFID tag, the data storage chip, etc.), the pump/catheter assembly 14, the thrombectomy device 58, the inflow pump 56, etc. to obtain identifying information related thereto which is associated with specifications and/or operational parameters stored in the memory of the user interface 32 and/or the controller 33. The identifying information may be used by the user interface 32 and/or the controller 33 to access the specifications and/or operational parameters associated with the pump/catheter assembly 14, the thrombectomy device 58, the inflow pump 56, etc. in use that is stored in the memory.

In some embodiments, the inflow pump 56 may include a hemispherically-shaped pump piston head 116 having a flexible boot 118 connected to and extending between the top body 114 and the pump piston head 116. In some instances, the lower portion 111 of the base 109 may serve as a mount for a first end of the bubble trap 60.

The connection manifold assembly 62 may be secured directly to a second end of the bubble trap 60 and in some instances may include a bracket 120 to which is attached a vertically oriented tubular manifold 148 having a plurality of ports attached or formed therethrough including a fluid (e.g., saline) inlet port 122, an effluent outlet port 124, a Luer style effluent return port 126, and/or an auxiliary port 128 and cap 130. Also shown are connectors 132 and 134 connectingly extending between the connection manifold assembly 62 and the upper portion 110 of the base 109.

The bubble trap 60 may include two mating halves of which a first bubble trap half 60a is shown. A hydrophobic filter 136 may be included at the upper forward region of the first bubble trap half 60a. In some embodiments, a second hydrophobic filter may be included on the second bubble trap half (not explicitly shown) which opposes the hydrophobic filter 136 on the first bubble trap half 60a.

The fixture 140, and components associated therewith, assists in support and connection of the effluent return tube 66 to the effluent return port 126 by a connector 142 combined continuously with a connection tube 144, and also assists in support, passage, and connection of the fluid supply tube 70 with the fluid inlet port 122. The fixture 140 may include outwardly extending vertically aligned and opposed tabs 141a and 141b which prevent the fixture 140 and associated effluent return tube 66 containing the high-pressure fluid supply tube 64 and the fluid supply tube 70 from contacting the roller pump 40 (e.g., FIG. 1) provided with the drive unit 12, such as located in the carriage assembly 22 or adjacent thereto.

In some embodiments, the effluent waste tube 68 may be positioned within and/or through the roller pump with the effluent collection bag 28 (e.g., FIG. 1) connected to the effluent waste tube 68. The effluent collection bag 28 may be suitably positioned for collecting effluent during the procedure. Pump rollers of the roller pump 40 may engage the effluent waste tube 68 to control effluent fluid flow through the effluent waste tube 68 from the effluent outlet port 124 to the effluent collection bag 28. In some embodiments, other pump types and/or configurations may be used in place of the roller pump.

At an appropriate time, the thrombectomy device 58 may be subjected to a priming procedure to purge the thrombectomy device 58 of any air. For example, the tip of the thrombectomy device 58 may be placed in a bowl of sterile saline, or other fluid, and the inflow pump 56 may be operated by action of the reciprocating linear actuator 84 (such as by activating and/or depressing the user activation switch 40) to prime the thrombectomy device 58. Thereafter, medical personnel may insert the thrombectomy device 58 into the vasculature of the patient, and operation of the thrombectomy system 10 incorporating the user interface 32 and the user activation switch 40 can begin, as desired. The reciprocating linear actuator 84 is actuated according to the operating parameters to influence fluid inflow pressures, pump speed, flow rates, and the like to operate the inflow pump 56 to deliver pressurized fluid to the thrombectomy device 58 via the high-pressure fluid supply tube 64 residing in the effluent return tube 66. Supply fluid is routed through the bubble trap 60, may be pressurized by the inflow pump 56, and is routed through the high-pressure fluid supply tube 64 to the thrombectomy device 58 for use in a thrombectomy or other related procedure. Effluent may be returned through the effluent return tube 66 to the connection manifold assembly 62 for collection in the effluent collection bag 28 (e.g., FIG. 1) through the effluent waste tube 68, which may be controlled by the roller pump.

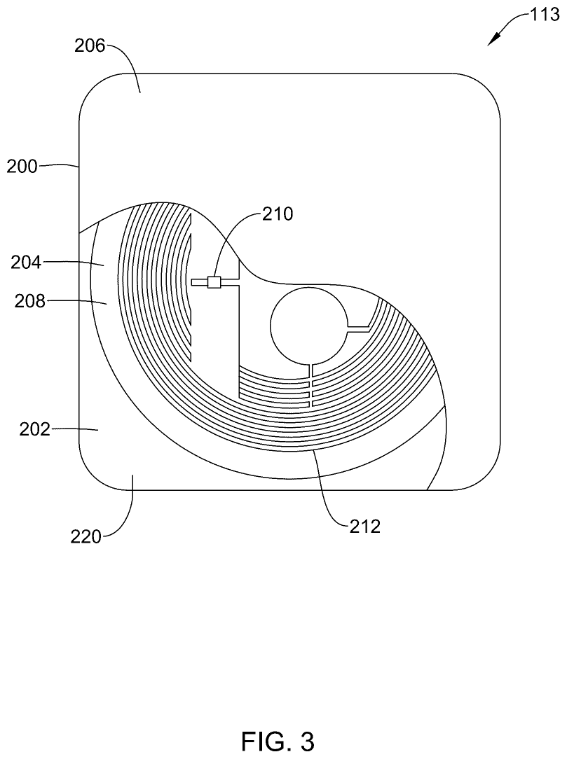

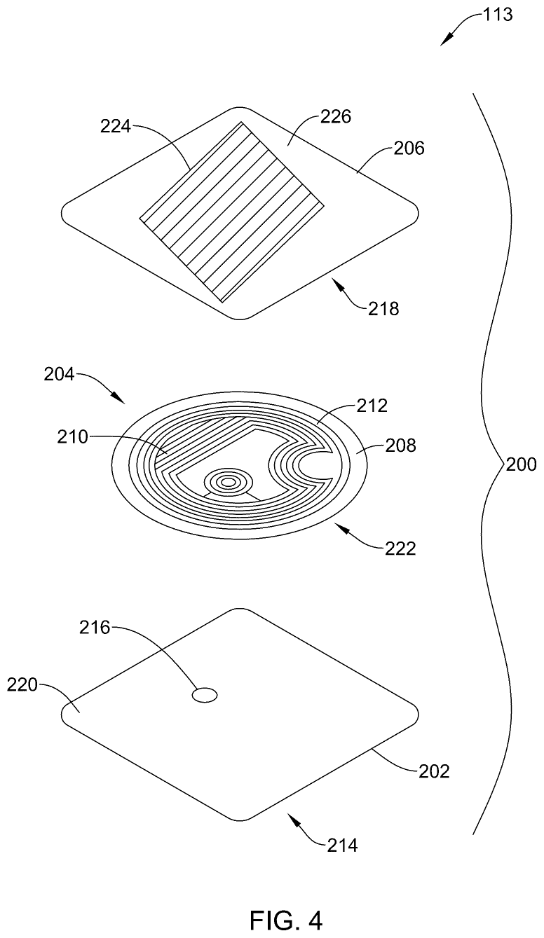

FIG. 3 illustrates a top view of an illustrative data plate 113 with a partial cut-out. FIG. 4 is an exploded perspective view of the illustrative data plate 113 of FIG. 3. In some examples, the data plate 113 may be provided as an adhesive label 200 that can be adhesively affixed to the pump/catheter assembly 14. The data plate 113 may include a radiofrequency identification (RFID) tag 204 disposed between a base layer 202 and a top layer 206. Generally, the RFID tag 204 may include a substrate 208, a chip 210, and an antenna 212. The chip 210 may be an integrated circuit (IC) having a memory and a processor, such as, a microprocessor or decision-making unit (logic). The antenna 212 may be electrically coupled to the chip 210. In some cases, the antenna 212 may be a strip of conductive material, such as, but not limited to, copper, aluminum, etc. The antenna 212 may be configured to receive the signals emitted from the data acquisition device 35 and reflect signals to the data acquisition device 35. In some cases, the antenna 212 may collect energy from the radio waves of the data acquisition device 35. This energy may be supplied to the chip 210 to power the chip 210. Such an RFID tag 204 may be considered to be a passive RFID tag 204. While the RFID tag 204 is described as a passive tag, in some cases, the RFID tag 204 may be an active tag. An active RFID tag 204 may include a battery (not explicitly shown) which powers the chip 210 and allows the antenna 212 to transmit signals to the data acquisition device 35. The substrate 208 may be formed from a thin, flexible polymeric material. The chip 210 and the antenna 212 may be secured to the substrate 208 to maintain the chip 210 and the antenna 212 in a predetermined orientation. The RFID tag 204 may have an average resonant frequency in the range of about 13.55 megahertz (MHz) to about 14.25 MHz. However, other resonant frequencies may be used, if so desired.

The base layer 202 of the adhesive label 200 may be a polyester label material having an acrylic adhesive disposed on a bottom surface 214 thereof. However, other label materials and/or adhesives may be used, as desired. The base layer 202 may further include an RFID compensation hole 216 extending through a thickness thereof. When the RFID tag 204 is assembled with the base layer 202, the chip 210 may be aligned with the RFID compensation hole 216. The RFID compensation hole 216 may provide a space for the chip 210 to rest within so that when the top layer 206 is disposed over the RFID tag 204 and the base layer 202, the top layer 206 is free from a bulge that may otherwise be caused by the chip 210. For example, the chip 210 may have a thickness greater than a remainder of the RFID tag 204 and absent a hole or recess for the chip 210 to be positioned within, a bump or bulge may be present in the top layer 206. In some cases, the RFID compensation hole 216 may be omitted. Prior to applying the label 200, including the RFID tag 204, to the pump/catheter assembly 14, the label 200 may be secured to a release liner (not explicitly shown) via the adhesive on the bottom surface 214 of the base layer 202. When it is desired to apply the label 200 including the RFID tag 204 to the thrombectomy catheter, the label 200 may be peeled or released from the release liner with the adhesive remaining on the bottom surface 214 of the base layer 202. The label 200 may then be adhesively secured to the pump/catheter assembly 14 via the adhesive on the bottom surface 214 of the base layer 202. In FIG. 2, the data plate 113 is illustrated as affixed to a body portion of the fluid inflow pump. However, the data plate 113 may be affixed to other portions of the pump/catheter assembly 14, as desired.

The top layer 206 of the adhesive label 200 may be a polyester label material having an acrylic adhesive disposed on a bottom surface 218 thereof. However, other label materials and/or adhesives may be used, as desired. When the label 200 is assembled, the RFID tag 204 may be positioned between the bottom surface 218 of the top layer 206 and a top surface 220 of the bottom layer 202. The RFID tag 204 may have a surface area less than the surface area of the bottom layer 202 and/or top layer 204. The RFID tag 204 to be secured to the bottom surface 218 of the top layer 206 via the adhesive thereon. The portion of the top layer 206 that is free from the RFID tag 204 may be secured to the top surface 220 of the base layer 202 to secure the three components 202, 204, 206 to one another. However, other coupling arrangements may be used as desired. For example, adhesive may be provided on a bottom surface 222 of the substrate 212 of the RFID tag 204 to secure to the RFID tag 204 to the base layer 202.

In some embodiments, an optional barcode 224, such as but not limited to, a linear barcode or matrix (2D) barcodes may be printed on a top surface 226 of the top layer 206 of the adhesive label 200. The barcode 224 may be scannable by a data acquisition device 35 to transfer information regarding the pump/catheter assembly 14, operating parameters thereof, etc. to the controller 33. It is contemplated that the presence of the barcode 224 and the RFID tag 204 may allow the data plate 113 to be used with a console 12 that includes either a barcode scanner or an RFID reader as the data acquisition device 35. However, a barcode 224 may provide less information and/or fewer operating parameters to the controller 33.

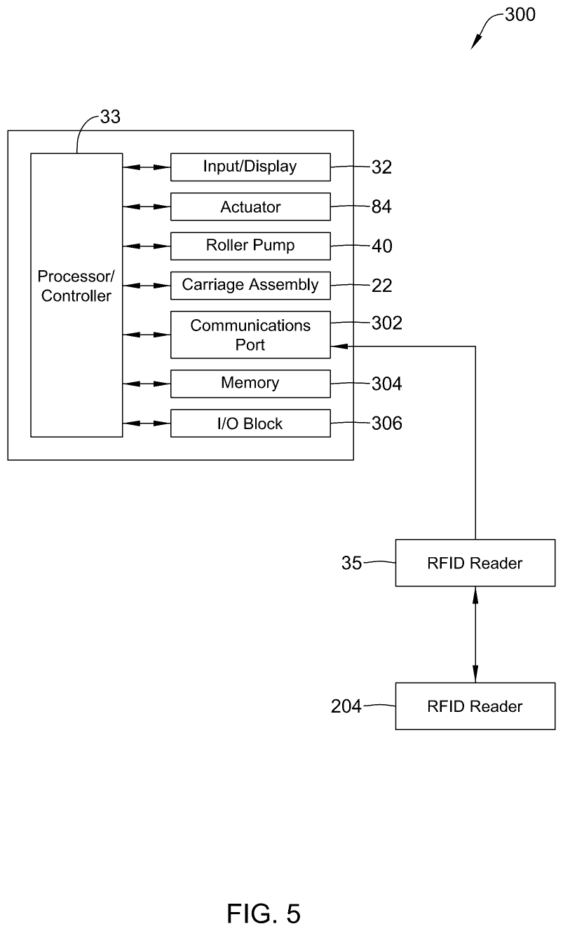

FIG. 5 is a schematic block diagram 300 of an illustrative control and communications system for the thrombectomy system 10. As described above, the console 12 may include a controller 33 and one or more controllable components, such as, but not limited to, the reciprocating linear actuator 84, the roller pump 40, the carriage assembly 22, etc. The controller 33 may be configured to send operational data to the controllable components. For example, the controller 33 may include control circuitry and logic configured to operate, control, command, etc. the various components (not explicitly shown) of the console 12, display data and/or operating parameters on the display 32, and/or issue alerts or notifications.

The controller 33 may be in communication with any number of controllable components as desired, such as, but not limited to, one, two, three, four, or more. The controller 33 may be configured to communicate with the controllable components using wired or wireless communications. The controller 33 may include a processor (e.g., microprocessor, microcontroller, etc.) and a memory 304. In some cases, the controller 33 may include a user interface 32 including a display and a means for receiving user input (e.g., touch screens, buttons, keyboards, etc.). The memory 304 may be in communication with the processor. The memory 304 may be used to store any desired information such as, but not limited to, control algorithms, configuration protocols, set points, authentication information, error conditions, etc.

In some embodiments, the controller 33 may include an input/output block (I/O block) 306 having a number of wire terminals for receiving one or more signals from the controllable components and/or for providing one or more control signals to the controllable components. For example, the I/O block 306 may communicate with one or more components of the console 12, including, but not limited to, the reciprocating linear actuator 84, the roller pump 40, the carriage assembly 22, etc. The controller 33 may have any number of wire terminals for accepting a connection from one or more components of the console 12. However, how many wire terminals are utilized and which terminals are wired is dependent upon the particular configuration of the console 12. In some cases, the I/O block 306 may be configured to receive wireless signals from the controllable components.

It is contemplated that the controller 33 may be connected to a remote computer or server using a local area network (LAN), a wide area network (WAN), the Internet, etc. This may allow the controller 33 to communicate with external databases to authenticate the pump/catheter assembly 14, check for prior use of the pump/catheter assembly 14, transmit operation logs, etc.

The controller 33 may be configured to be in communication with the data acquisition device 35 via a communications port 302. It is contemplated that the communication port 302 may be in wired or wireless communications with the data acquisition device 35. In some embodiments, the data acquisition device 35 may be an RFID reader configured to periodically transmit an electromagnetic interrogation pulse. The console 12 may include a proximity sensor 37 (e.g., FIG. 1) for the pump/catheter assembly 14 such that when the pump/catheter assembly 14 is positioned within the carriage assembly, the RFID reader 35 is activated and begins to transmit the interrogative pulse. In response to the interrogative pulse(s), the RFID tag 204 may transmit data from the chip 210 to the RFID reader 35. The RFID reader 35 may in turn transmit the data received from the chip 210 to the controller 33. The controller 33 may then use this received data to operate the console 12, validate the pump/catheter assembly 14, perform safety checks, etc. It is contemplated that the use of a proximity sensor to activate the RFID reader 35 may help prevent the RFID reader 35 from receiving information from a different RFID tag that may be in close proximity to the RFID reader 35 or from reading the RFID tag 204 prematurely.

It is contemplated that an RFID tag 204 may be configured to store more data than a barcode 224. For example, while a barcode 224 may be limited to a predetermined number of characters, an RFID tag 204 can store in the range of about 2 kilobytes of information. High capacity RFID tags 204 may store in the range of about 4-8 kilobytes of information. Further, compared to barcodes 224 an RFID tag 204 may include more programmable fields and may be easier to program during production. As such, the use of an RFID tag 204 to store and transmit data to the controller 33 may improve the functionality of the console 12. Further, as the RFID tag 204 communicates using wireless protocols, the RFID tag 204 may continue to function even if the data plate 113 or the barcode 224 (if so provided) gets dirty, smears, etc. The barcode 224 may be provided with the RFID tag 204 to allow the data plate 113 to be backwards compatible with older models of the console 12 that may not necessarily include an RFID reader 35.

In some examples, the RFID tag 204 may include operational parameters for the reciprocating linear actuator 84. More specifically, the RFID tag 204 may store parameters which allow the speed of the reciprocating linear actuator 84 to be variable and in some cases, controlled by the operator. For example, the RFID tag 204 may contain parameters that when transmitted to the controller 33 cause the controller 33 to display a menu on the display 32 which allows the user to selectively adjust (e.g., increase and/or decrease) the speed of the reciprocating linear actuator 84. The menu may include one or more user selectable operating speeds for the reciprocating linear actuator 84 and/or up/down inputs to allow a user to incrementally adjust the speed of the reciprocating linear actuator 84. This may allow the user to increase or decrease the thrombectomy action as needed or desired. In some cases, the menu may allow the user to select the age of the thrombus (e.g., acute, subacute, and/or chronic). The controller 33 may then select the speed of the reciprocating linear actuator 84 based on the age of the thrombus to allow for more vigorous or less vigorous thrombectomy action

In another example, the RFID tag 204 may store parameters configured to drive the reciprocating linear actuator 84 with a varying speed curve. It is contemplated that the speed of the reciprocating linear actuator 84 may vary over each stroke or over a period of time. For example, within each stroke of the reciprocating linear actuator 84, a fast-pulsatile action may create a frequency that helps to fragment and remove thrombus or other undesirable material. When the RFID tag 204 transmits data to the controller 33 that includes a varying speed curve, the controller 33 may control the reciprocating linear actuator 84 at different speeds over the length of a procedure. It is contemplated that the speed at which the reciprocating linear actuator 84 is operated may increase as time passes as increased procedure time may indicate a tougher thrombus. The speed of the reciprocating linear actuator 84 may vary in a number of different manners. For example, the speed reciprocating linear actuator 84 may increase in a linear manner, the speed may increase rapidly over a first time duration and then may increase more slowly over a second time duration longer than the first, the speed may increase slowly over a first time duration and then increase rapidly over a second time duration less than the first time duration, etc. These are just some examples. Other speed curves or profiles may be used, as desired. In some cases, during a procedure, more vigorous action (e.g., increased speed of the reciprocating linear actuator 84) and/or the longer the pump/catheter assembly 14 is in use may be indicative of a tougher clot.

In yet another example, the RFID tag 204 may store parameters that allow the controller 33 to adjust a stroke length and/or frequency of the reciprocating linear actuator 84. It is contemplated that rapid, short strokes may enhance a fast-pulsatile frequency. The RFID tag 204 may contain parameters that when transmitted to the controller 33 cause the controller 33 to display a menu on the display 32 which allows the user to adjust the stroke length of the reciprocating linear actuator 84 to provide better control over the infusion volume of a user specified fluid, drug, or therapeutic agent. In yet other examples, the RFID tag 204 may transmit operational parameters to the controller 33 which cause the controller 33 to control the reciprocating linear actuator 84 at a fixed stroke length specific to the particular pump/catheter assembly 14 in use. For example, some thrombectomy catheters 14 may be more powerful or less powerful than others which may require different stroke lengths of the reciprocating linear actuator 84.

In some examples, the RFID tag 204 may include operational parameters for the roller pump 40. In some cases, the RFID tag 204 may include one or more flags to indicate a mode of operation for the roller pump 40. One illustrative operational mode may force the pump/catheter assembly 14 into a permanent power pulse mode. Power pulse mode may be the mode of operation in which the system 10 infuses (without removing) a physician-specified fluid or therapeutic agent. Permanent power pulse mode may lock a pump/catheter assembly 14 into a power pulse mode and precludes or prevents the system 10 from switching to a removal (e.g., thrombectomy) mode that extracts fluid and clot from the patient. Another illustrative operational mode may accommodate differing physical roller pumps. For example, different consoles 12 may have different roller pumps 40. The RFID tag 204 may provide operational modes for the differing roller pumps 40 and the controller 33 may then control the particular roller pump 40 based on the parameters provided for that roller pump 40 by the RFID tag 204.

In another example, the RFID tag 204 may provide parameters that allow the roller pump 40 to run in an intermittent manner instead of continuously. More specifically, the RFID tag 204 may provide parameters to the controller 33 that allow the controller 33 to turn on the roller pump 40 every other minute, or at some other predetermined time duration. It is further contemplated that the RFID tag 204 may provide parameters to the controller 33 that allow the controller 33 to turn on the roller pump 40 based on a number of strokes of the reciprocating linear actuator 84. For example, the controller 33 may turn on the roller pump 40 with every five strokes of the reciprocating linear actuator 84, or another stroke frequency. Reducing the active time of the roller pump 40 may help reduce effluent flow and improve fluid balance.

In yet another example, the RFID tag 204 may provide parameters that allow the roller pump 40 to run in reverse. More specifically, the RFID tag 204 may provide parameters to the controller 33 that allow the controller 33 to operate the roller pump 40 in reverse to actuate an accessory, such as, but not limited to, opening or closing a valve by pressure provided from the roller pump 40. In another example, the RFID tag 204 may provide parameters to the controller 33 that allow the controller 33 to operate the roller pump 40 in reverse to inject contrast during use of the pump/catheter assembly 14. In some cases, the user may provide an input to the controller 33 via the user interface 32 to indicate a desire to operate the roller pump 40 in reverse. In other cases, the controller 33 may be configured to automatically operate the roller pump in reverse in response to one or more operating conditions.

In yet another example, the RFID tag 204 may provide parameters that allow the roller pump 40 to operate independently from the reciprocating linear actuator 84. For example, the RFID tag 204 may provide parameters to the controller 33 that allow the controller 33 to operate the roller pump 40 when the reciprocating linear actuator 84 is stationary to allow the roller pump 40 to function as an infusion pump to provide fluid to the body. In some cases, the RFID tag 204 may provide parameters to the controller 33 that allow the user to adjust the operational settings of the roller pump 40. For example, the RFID tag 204 may provide parameters to the controller 33 that cause the controller 33 to display on the user interface one or more user selectable or adjustable operating parameters of the roller pump 40. Upon receiving the selection from the user, the controller 33 may then control the roller pump 40 in accordance with the user selections.

In some examples, the RFID tag 204 may provide parameters that allow the controller 33 to calculate desired operating conditions. For example, the RFID tag 204 may provide parameters to the controller 33 that allow the controller 33 to calculate the correct pressure based on a diameter of the pump piston head 116. This may allow the use of thrombectomy catheters 14 having varying sizes of piston diameters. The RFID tag 204 may be configured to provide other physical attributes of the pump/catheter assembly 14, as desired.

It is further contemplated that the RFID tag 204 may provide parameters to the controller 33 that allow the controller 33 to authenticate the pump/catheter assembly 14. For example, the RFID tag 204 may provide a serial number or unique identification number to the controller 33. The controller 33 may be configured to reference a database of previously used thrombectomy catheters 14 to verify the particular pump/catheter assembly 14 currently in the console 12 has not been previously used. Once the controller 33 verifies the particular pump/catheter assembly 14 has not been used, the controller 33 may be configured to update the database to include the serial number or unique identification number to the particular pump/catheter assembly 14 that is currently in use to prevent the reuse of said pump/catheter assembly 14. If the controller 33 determines that the pump/catheter assembly 14 has already been used in a different patient or at another time, the controller 33 may be configured to display an alert or error message on the user interface 32 indicating the pump/catheter assembly 14 should not be used. It is further contemplated that the controller 33 may prevent the operation of the console 14 while a pump/catheter assembly 14 that has been indicated as previously used remains within the carriage assembly 22. Additionally, or alternatively, the controller 33 may be configured to reference a database of valid devices (e.g., serial numbers, models, other unique identifier) for use in the console 12. This may help ensure unauthorized and/or pirated devices are not used with the console 12 which may create unsafe conditions. If the controller 33 determines that the pump/catheter assembly 14 should not be used with the console 12, the controller 33 may be configured to display an alert or error message on the user interface 32 indicating the pump/catheter assembly 14 should not be used.

In some cases, the console 12 and/or controller 33 may be connected to the Internet. The RFID tag 204 may include identifying information, such as, but not limited to, a serial number, a unique identification number, a model type, etc. that may be transmitted to the controller 33. The controller 33 in response to receiving the identifying information may be configured to poll a database or server for warnings or alerts associated with the particular pump/catheter assembly 14. This may include, but is not limited to, recall alerts for a pump/catheter assembly 14 that has been positioned within the console 12.

In yet other examples, the RFID tag 204 may include parameters that vary the response of the controller 33 to possible error conditions. For example, the RFID tag 204 may provide parameters to the controller 33 that allow the controller 33 to attempt a pre-determined number of self-corrections in response to an error condition before triggering an error condition alert or message. Self-corrections may include, but are not limited to reading other parameters from the RFID tag 204, rereading the parameters from the RFID tag 204, retrying pump priming, etc. The pre-determined number of self-corrections and/or the self-correction action may vary based on the pump/catheter assembly 14, the error condition, etc.

It is further contemplated that the RFID tag 204 may include parameters that allow the controller 33 to display information on the user interface 32 specific to the particular pump/catheter assembly 14. For example, the RFID tag 204 may provide parameters to the controller 33 that allow the controller 33 to display on the user interface 32 warnings or cautions associated with the use of certain models of the pump/catheter assembly 14. In another example, the RFID tag 204 may provide parameters to the controller 33 that allow the controller 33 to display on the user interface recommended usage techniques for the particular pump/catheter assembly 14. It is further contemplated that the RFID tag 204 may provide parameters to the controller 33 that allow the controller 33 to display on the user interface marketing information for the particular pump/catheter assembly 14.

In further examples, the RFID tag 204 may include parameters that further improve the safety of the thrombectomy system 10. In some cases, the RFID tag 204 may include parameters that prevent unsafe conditions due to extended use of the pump/catheter assembly 14 or use of the pump/catheter assembly 14 beyond a predetermined length of time. For example, the RFID tag 204 may provide parameters, such as recommended run time limits to the controller 33. The controller 33 may then display alerts to the user via the user interface 32 when the run time limit is approaching, has been met, and/or has been exceeded. In some cases, the RFID tag 204 may provide parameters to the controller 33 such that the controller triggers a hard stop of the console 12 if run time limits have been met or exceeded. Additionally, or alternatively, the RFID tag 204 may include parameters the allow the controller 33 to display on the user interface 32 other important limits and/or information, such as, but not limited to, the estimated blood loss from a procedure.

For convenience, the user could program limits (such as, but not limited to, procedure time, blood loss, other parameters, etc.) in response to achieving the controller 33 may automatically stop operation of the console or give an audible or visual alert (e.g., at the user interface 32). This may obviate the need for the user to watch the user interface 32. The procedure could be restarted after the stop. The RFID tag 204 may include read/write controls to prevent the RFID tag 204 from being altered after the time of manufacture. However, in some cases, with the right permissions the RFID tag 204 may be programmed in the field to allow the RFID tag 204 to transmit such control limits and/or other operational parameters to the controller 33.

In some examples, the RFID tag 204 may include parameters that improve quality monitoring, quality control, and/or complaint monitoring. For example, the RFID tag 204 may include control parameters that when transmitted to the controller 33 cause the controller 33 to log usage information regarding the pump/catheter assembly 14 and/or the console 33. For example, the controller 33 may log run times, stroke lengths, stroke speed, roller pump data, infusion rates, effluent rates, error conditions, etc. This data may be correlated to the specific pump/catheter assembly 14 and the specific console used and transmitted via the Internet or other network to a database or external server. User complaints that are received can be evaluated against the specific pump/catheter assembly 14 and/or console 12 used. This may help determine whether an incorrect complaint has been filed or identify trends in complaints. The data may be analyzed individually or with the usage data from other devices and/or procedures to more accurately track and troubleshoot error conditions and user complaints. In addition to tracking error conditions and user complaints, the data may be used to track effectiveness to changes to the pump/catheter assembly 14 and/or the operating parameters thereof. For example, it may be desirable to track whether or not changes improve the treatment outcome such as by reducing a treatment time or reducing error conditions.

The RFID tag 204 may contain text translations for languages that are not currently programmed into the controller 33 and/or console 12. The RFID tag 204 may provide parameters to the controller 33 that allow the controller 33 to display to the user on the user interface 32 a language selection menu. Upon selection of a desired language, the controller 32 may then display all commands, prompts, data, errors, etc. in the selected language. The RFID tag 204 may contain different sounds and/or tones for auditory alerts. The RFID tag 204 may provide parameters to the controller 33 that allow the controller 33 to display to the user on the user interface 32 a sound and/or tone selection menu providing a plurality of distinct sounds and/or tones. Upon selection of a desired sound and/or tone from the plurality of distinct sounds and/or tones, the controller 33 may then use the selected sound and/or tone for future auditory alerts. For example, selection of the sound and/or tone by the user may assign the selected sound and/or tone to a selected alert and/or notification or all alerts and/or notifications. In some cases, the RFID tag 204 may be used for inventory tracking. For example, RFID readers separate from those provided on the console 12 may be configured or used to track a pump/catheter assembly 14 at a distribution center, hospital, clinic, etc. to allow a responsible party to track a number and/type of pump/catheter assembly 14 at a particular location.

The materials that can be used for the various components of the thrombectomy catheter, pump/catheter assembly, and/or other devices disclosed herein may include those commonly associated with medical devices. For simplicity purposes, the following discussion makes reference to the pump/catheter assembly and its related components. However, this is not intended to limit the devices and methods described herein, as the discussion may be applied to other similar devices, tubular members and/or components of tubular members or devices disclosed herein.

The various components of the devices/systems disclosed herein may include a metal, metal alloy, polymer (some examples of which are disclosed herein), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-clastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In at least some embodiments, portions or all of the pump/catheter assembly and its related components may be doped with, made of, or otherwise include a radiopaque material. Radiopaque materials are understood to be materials capable of producing a relatively bright image on a fluoroscopy screen or another imaging technique during a medical procedure. This relatively bright image aids the user of the pump/catheter assembly and its related components in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the pump/catheter assembly and its related components to achieve the same result.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A thrombectomy system, the system comprising:
a console including a radiofrequency identification (RFID) reader;
a fluid inflow pump, the fluid inflow pump driven by the console;
a thrombectomy catheter, the fluid inflow pump configured to provide fluid inflow through the thrombectomy catheter; and
an RFID tag coupled to a body portion of the fluid inflow pump;
wherein when the fluid inflow pump is positioned within the console, the RFID reader is configured to receive one or more operating parameters from the RFID tag.

2. The thrombectomy system of claim 1, further comprising a proximity sensor.

3. The thrombectomy system of claim 2, wherein the proximity sensor is configured to detect the fluid inflow pump is positioned within the console and activate the RFID reader.

4. The thrombectomy system of claim 1, wherein the RFID reader is configured to transfer the one or more operating parameters to a controller of the console.

5. The thrombectomy system of claim 4, wherein in response to receiving the one or more parameters the controller is configured to control one or more components of the thrombectomy system according to the one or more operating parameters.

6. The thrombectomy system of claim 4, wherein in response to receiving the one or more parameters the controller is configured to vary an operational speed of a reciprocating linear actuator.

7. The thrombectomy system of claim 4, wherein in response to receiving the one or more parameters the controller is configured to display a menu on a user interface, the menu including one or more user selectable operating parameters.

8. The thrombectomy system of claim 4, wherein in response to receiving the one or more parameters the controller is configured to display a menu on a user interface, the menu including one more user selectable sounds and/or tones to be assigned by the user for a selected alert and/or notification.

9. The thrombectomy system of claim 4, wherein in response to receiving the one or more parameters the controller is configured to display on a user interface information specific to the thrombectomy catheter and fluid inflow pump.

10. The thrombectomy system of claim 4, wherein in response to receiving the one or more parameters the controller is configured to vary a stroke length of a reciprocating linear actuator of the console.

11. The thrombectomy system of claim 4, wherein in response to receiving the one or more parameters the controller is configured to intermittently activate a roller pump of the console.

12. The thrombectomy system of claim 4, wherein in response to receiving the one or more parameters the controller is configured to reverse an operational direction of a roller pump of the console.

13. The thrombectomy system of claim 4, wherein in response to receiving the one or more parameters the controller is configured to authenticate the fluid inflow pump and thrombectomy catheter.

14. The thrombectomy system of claim 4, wherein in response to receiving the one or more parameters the controller is configured to log usage information within the controller and/or record and transmit operational data to a remote device.

15. The thrombectomy system of claim 1, wherein the RFID tag is disposed within an adhesive label.

16. A thrombectomy system, the system comprising:
a console including a radiofrequency identification (RFID) reader;
a fluid inflow pump, the fluid inflow pump driven by the console;
a thrombectomy catheter, the fluid inflow pump configured to provide fluid inflow through the thrombectomy catheter; and
a data plate coupled to a body portion of the fluid inflow pump, the data plate comprising:
a base layer including an adhesive on a bottom surface thereof;
a top layer; and
a radiofrequency identification (RFID) tag disposed between the base layer and the top layer;
wherein when the fluid inflow pump is positioned within the console, the RFID reader is configured to receive one or more operating parameters from the RFID tag.

17. A data plate for a thrombectomy system, the data plate comprising:
a base layer including an adhesive on a bottom surface thereof;
a top layer;
a radiofrequency identification (RFID) tag disposed between the base layer and the top layer; and
a barcode formed on a top surface of the top layer.

18. The data plate of claim 17, wherein the top layer is adhesively secured to the RFID tag and a top surface of the base layer.

19. The data plate of claim 17, wherein the base layer and the top layer are formed from a polyester label material.

20. The data plate of claim 17, wherein the base layer includes an RFID compensation hole extending through a thickness thereof, the RFID compensation hole aligned with a chip of the RFID tag.
